(19) United States
(12) Patent Application Publication
(10) Pub. No.: US 2010/0151502 A1
(43) Pub. Date: Jun. 17, 2010

(54) FUNCTIONAL EXPRESSION OF TRIACYLGYLCEROL LIPASES

(75) Inventors: Bernhard Hauer, Fussgonheim (DE); Dietmar Häring, Neu-Edingen (DE); Cecilia Kvarnstroem-Branneby, Stockholm (SE); Rolf Schmid, Stuttgart (DE); Monika Müller, Aachen (DE); Danni Liu, Stuttgart (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/295,889

(22) PCT Filed: Apr. 5, 2007
(86) PCT No.: PCT/EP2007/053416

§ 371 Date: Mar. 2, 2010

(30) Foreign Application Priority Data
Apr. 5, 2006 (DE) 10 2006 016 023.1

(51) Int. Cl. G01N 33/554; C12N 9/20; C07H 21/00; C12N 15/74; C12N 1/21

(52) U.S. Cl. 435/7.32; 435/198; 536/23.2; 435/320.1; 435/252.3

(57) ABSTRACT

The present invention relates to nucleic acids that code for triacylglycerol lipases, vectors comprising said nucleic acids, host cells comprising said nucleic acids or vectors, methods for the expression of triacylglycerol lipases in prokaryotes, methods for the detection and for the production of triacylglycerol lipases, triacylglycerol lipases obtainable thereby, and the use of triacylglycerol lipase-encoding nucleic acids, vectors and recombinant host cells for said methods.

The present invention relates to nucleic acids that encode triacylglycerol lipases, vectors comprising said nucleic acids, host cells that comprise said nucleic acids or vectors, methods of expression of triacylglycerol lipases in prokaryotes, methods for the detection and for the production of triacylglycerol lipases, triacylglycerol lipases obtainable thereby, and the use of triacylglycerol lipase-encoding nucleic acids, vectors and recombinant host cells for the aforesaid methods.

### BACKGROUND OF THE INVENTION

Triacylglycerol lipases (EC 3.1.1.3) are valued, efficient catalysts for a great variety of industrial uses, for example in the detergents industry, oil chemistry, the food industry and in the production of fine chemicals (Schmid 1998). Lipases are carboxylic ester hydrolases, which catalyze both the hydrolysis and the synthesis of triglycerides and other generally hydrophobic esters. All triacylglycerol lipases, whose three-dimensional crystal structure has been elucidated, belong to the α/β-hydrolase folding protein family, which have a similar overall architecture (Ollis 1992).

Candida antarctica-lipase B (CalB) is an efficient catalyst for many reactions and is used for example for stereoselective transformations and polyester synthesis (Anderson 1998). CalB has a solvent-accessible active center (Uppenberg 1994) and does not display interphase activation (Martinelle at al., 1995). The active center is a narrow funnel and for this reason CalB has a higher activity with respect to carboxylic acid esters, for example ethyl octanoate, than with respect to triglycerides (Martinelle 1995). The fact that the activity of CalB in organic media is comparable to that in water, and in particular the high enantioselectivity of CalB for secondary alcohols make this enzyme one of the most important lipases currently in use in biotechnology.

In the past, for large-scale industrial applications CalB was mainly expressed in Aspergillus oryzae (Hoegh 1995). For research purposes the enzyme was expressed successfully in the yeasts Pichia pastoris (Rotticci-Mulder et al. 2001) and Saccharomyces cerevisiae (Zhang et al. 2003). Expression of CalB in the easily manageable prokaryotic expression system Escherichia coli (E. coli) was not successful (Rotticci-Mulder 2003). Expression in E. coli was achieved for the first time later, but only led to low yields of functional CalB (Rusnak 2004). This is unfortunate, as E. coli has many significant advantages over other expression systems and permits rapid and inexpensive high-throughput screening of large gene libraries.

Modification of CalB by random mutagenesis was described recently (Chodorge et al., 2005). Several attempts to improve CalB for special applications through rational enzyme design have also been reported in the literature. Although some of these led to good results (Patkar at al. 1998; Rotticci 2000), the possibilities for rational enzyme design are still limited through insufficient understanding of the catalytic properties of the enzyme.

However, the main problem that has yet to be solved is the inadequate functionality of CalB on expression in E. coli, which is a prerequisite for the improvement of enzymes through directed evolution. The reason for this is considered to be the complex tertiary structure of the enzyme, which requires the formation of three disulfide bridges in order to ensure functional conformation. There are difficulties in producing such a protein in E. coli or other prokaryotes, because the cellular environment, the folding machinery and the checkpoints of folding quality control of prokaryotes differ from those of the eukaryotes (Baneyx and Mujacic 2004). Correspondingly, in initial expression experiments of CalB in E. coli the inventors found there was formation of inclusion bodies and lack of activity of CalB (results not shown).

Additional problems can occur at the translation level. The quantity of tRNA species can vary considerably in the various organisms. This problem can be overcome by codon optimization, and in fact the expression levels of some eukaryotic proteins, e.g. a domain of the human type 1 neurofibromin protein, were raised significantly (Hale 1998). However, the amount of functional enzyme is not directly correlated with the expression level and therefore is also only conditionally correlated with codon usage.

In addition to the actual gene sequence, the promoter plays a central role in expression efficiency. In biotechnology, vector systems that are often used, e.g. the pET vector system (Novagen), contain the T7 promoter, which makes them suitable for strict regulation of the pronounced overexpression of heterologous proteins in E. coli. However, high expression levels of heterologously expressed enzymes very often lead to incorrectly folded proteins.

It had been shown that cold-sensitive promoters can facilitate efficient gene expression of certain proteins at reduced temperatures. In particular, the promoter of the principal cold-shock gene cspA had been used in the past (Goldstein et al. 1990). As is clear from comparative studies, however, expression of soluble protein in E. coli is still problematic and depends on the protein and on the organism from which the protein originates (Qing et al. 2004).

The cellular environment can also exert an influence on the yield of functional, i.e. enzymatically active protein. It had been reported that mutations in the genes of glutathione reductase (gor) and thioredoxin reductase (trxB) can lead to increased formation of disulfide bridges in proteins on expression in the cytoplasm of E. coli (Prinz et al. 1997).

One approach for improving the yield of soluble proteins in the cytoplasm of E. coli comprises the co-expression of molecular chaperones, which are involved in de novo protein folding. Thus, it had been reported in the past that overexpression of the chaperones DnaK-DnaJ or Trigger Factor (TF) increases the solubility of selected proteins on expression in E. coli (Nishihara et al. 2000). Good results were reported for target proteins >60 kD. Another mechanism based on GroEL-GroES can be useful for target proteins that are smaller than about 60 kD (Baneyx and Mujacic 2004).

Despite the progress made in the past with respect to the functional expression of heterologous proteins in E. coli, successful expression still cannot be predicted, but depends considerably on the protein used in each case.

There are still no reports on increase in functional expression of recombinant triacylglycerol lipase, such as CalB in particular.

The aim of the present invention was therefore to provide nucleic acids that encode triacylglycerol lipases and methods for their improved functional expression in prokaryotes. Another aim was to provide a method of detecting triacylglycerol lipases, a method for the screening of said triacylglycerol lipases and methods of production of said triacylglycerol lipases.

### BRIEF DESCRIPTION OF THE INVENTION

The aim of the invention was achieved with a method of expression of triacylglycerol lipases, in which increased functional expression of the proteins is achieved by expression in prokaryotic, in particular E. coli host strains, under the special conditions described in more detail below.

The aim was also achieved by the provision of coding nucleotide sequences that are optimized with respect to the expression of lipase B in prokaryotes, in particular E. coli.

The aim was also achieved by a method of detecting triacylglycerol lipases, in particular CalB, the use of the method of detection for the screening of triacylglycerol lipases, and a method of production thereof.

### DESCRIPTION OF THE DRAWINGS

FIG. 1: sequence comparison between calB_wt amplified from C. antarctica and the synthetic sequence-optimized gene calB_syn originating from the pPCR/calB vector. Differences are highlighted.

FIG. 2: SDS-PAGE separation of soluble (S) and insoluble (I) fractions that were obtained at 15° C. using the expression vectors pET32-b(+) (in Origami™ 2 (DE3) cells) or pColdIII (in Origami™ B cells) in the CalB-expression experiments. CalB bands (33 kDa) and Trx-CalB fusion protein bands (45 kDa) are arrowed. M: molecular weight standard. C: fractions of a control with empty vector.

FIG. 3: SDS-PAGE separation of soluble (S) and insoluble (I) fractions that were obtained by co-expression experiments of CalB using pColdIII constructs with different chaperone plasmids (a: pGro7, b: pG-Tf2, c: pTf16, d: pKJE7, e: pG-KJE8) in Origami™ B cells. CalB (33 kDa) and chaperones (GroEL: 60 kDa, Tf: 56 kDa, DnaK: 70 kDa, DnaJ: 40 kDa) are arrowed. M: molecular weight standard (29, 43 and 66 kDa). C: fractions from a control with empty vector.

FIG. 4: Hydrolytic activity with respect to tributyrin of clarified cell lysates from E. coli Origami™ 2(DE3) cells (in the case of pET32b(+) expression) and Origami™ B cells (all other constructs), which bear the stated constructs. The mean value and the standard deviation from 4-6 independent expression experiments are shown.

FIG. 5: Hydrolysis of pNPP in 96-well microtiter plates by clarified cell lysates from Origami B cells, which contain pColdIII/calB_wt or _syn) and GroES/GroEL (pGro7). In the case of CalB-expressing cells, 17 (calB_wt) and 18 (calB_syn) wells were investigated. 6 wells with Origami™ B cells, which contained the empty pColdIII vector and pGro7, were used as controls. The values were normalized with background extinction values (substrate without cell lysate).

### DETAILED DESCRIPTION OF THE INVENTION

A first object of the invention relates to a method of expression of functional triacylglycerol lipase in prokaryotes, by expressing a triacylglycerol lipase-encoding nucleotide sequence in a prokaryotic cell, preferably in E. coli, under the control of an inducible promoter. Expression takes place in particular in a recombinant prokaryotic cell.

According to the invention, “triacylglycerol lipases” means enzymes of class E.C. 3.1.1.3 according to the IUBMB enzyme nomenclature (http://www.iubmb.unibe.ch; http://www.chem.qmul.ac.uk/iubmb/enzyme/). The method according to the invention is moreover suitable, in particular, for the functional expression of lipases that require, in their functional form, one or more S—S bridges (disulfide bridges), for example 1, 2, 3, 4, 5 or 6 S—S bridges per peptide chain, wherein the S—S bridges can be formed between sulfur-containing amino acids of the same peptide chain (intramolecular) and/or sulfur-containing amino acids of different peptide chains (intermolecular). Further examples of lipases with S—S bridges are lipase from Aspergillus oryzae (Tsuchiya et al., 1996), lipase from Penicilium camenbertii (Yamaguchi et al., 1991), lipase from Rhizomucor mihei (Boel et al., 1988) and lipase from Candida rugosa (Longhi et al., 1992).

In a special embodiment, expression takes place at low temperatures. For the purposes of this invention, low temperatures are understood to be room temperature or temperatures below room temperature, i.e. temperatures of about 25° C. or less, for example 25° C., 24° C., 23° C., 22° C., 21° C., 20° C., 19° C., 18° C., 17° C., 16° C., 15° C., 14° C., 13° C., 12° C., 11° C., 10° C., 9° C., 8° C., 7° C., 6° C., 5° C., 4° C., 3° C., 2° C., 1° C., 0° C., or temperatures between these values.

According to further embodiments, the temperature for expression is selected from a range from 1° C. to 20° C., in particular a range from 10° C. to 20° C., for example 10° C., 11° C., 12° C., 13° C., 14° C., 15° C., 16° C., 17° C., 18° C., 19° C. or 20° C., and in particular a range from 13° C. to 16° C., for example 13° C., 14° C., 15° C. or 16° C. In a special embodiment the temperature used for expression is about 15° C.

One object of the invention relates to expression in a thioredoxin-reductase-deficient and/or glutathione-reductase-deficient E. coli strain. Examples of said strains are Origami™ 2((DE3) and Origami™ B (Novagen, Darmstadt, Germany).

Without being bound to a theory, it is assumed that these enzymes prevent the formation of S—S bridges in proteins or contribute to the reduction of S—S bridges that have already formed. The absence of one or more of these enzymes or the suppression of their enzymatic activity therefore stabilizes the conformation of proteins containing said S—S bridges.

According to a special embodiment of the method according to the invention, the functionally expressed triacylglycerol lipase is lipase B, the gene product of CalB from Candida antarctica. The calB gene was described (Uppenberg et al., 1994) and its nucleotide or protein sequence was deposited under the access numbers 230645 and CAA83122.1 at GenBank. Unless designated more precisely, here CalB means a nucleotide sequence with this access number. Another example of a triacylglycerol lipase is lipase B from Pseudozyma tsukubaensis (Suen et al. 2004).

According to another special embodiment of the method according to the invention, the sequence coding for triacylglycerol lipase is calB_wt (SEQ ID NO:2). calB_wt originates from a previous project of the inventors, in which CalB was expressed functionally in Pichia pastoris, and is contained in the pPICZαA/calB construct (Rusnak 2004). In that project, the calB gene amplified from genomic DNA of Candida antarctica displayed two changes relative to the published CalB sequence (CAA83122.1) at the amino acid level (T57A, A89T; SEQ ID NO:13). The two deviations appeared in two independent amplification assays, in which the gene was amplified from two different extracts of genomic DNA. For this reason they are most probably natural variations of the lipase gene. The lipase showed, on expression in Pichia pastoris, an activity comparable to the published values of the wild-type CalB, so that in the previous projects the inventors continued the work with the amplified gene (Rotticci-Mulder et al., 2001; Rusnak 2004).

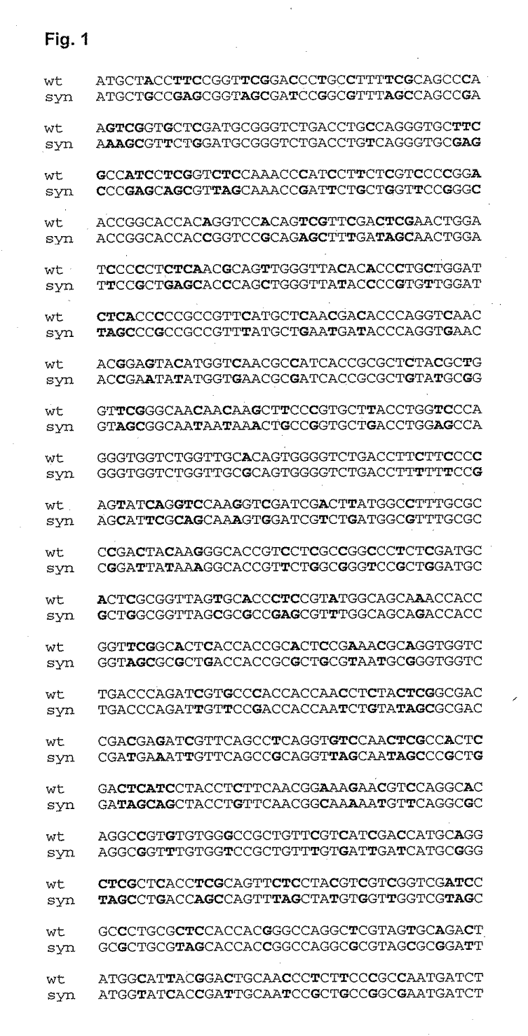

According to another special embodiment of the method according to the invention, the sequence coding for triacylglycerol lipase is calB_syn (SEQ ID NO:1). calB_syn resulted from sequence optimization. Sequence optimization strategies are known by a person skilled in the art and can comprise one or a combination of several measures. For example, codons are selected for amino acids so that they correspond to the transfer RNAs occurring relatively most frequently in the selected host. Furthermore, it may be advantageous to avoid regions with very high (>80%) or low (<30%) GC content or particular sequence motifs, which have an influence on the expression, and thus the transcription of the DNA and/or the translation of the mRNA. For the production of calB_syn, codon usage was optimized using the GeneOptimizer™ technology (GeneArt, Regensburg, Germany) and in addition regions with very high (>80%) or low (<30%) GC content were avoided if possible. Furthermore, cis-acting sequence motifs, for example internal TATA boxes, Chi sites, ribosomal linking sites, ARE, INS and CRS sequence elements as well as repetitive sequences and RNA secondary structures, were avoided. The gene differs in 253 nucleotides (26.5%) from the calB_wt sequence (FIG. 1). At the amino acid level the synthetic gene encodes the published protein (CAA83122.1; SEQ ID NO:12).

According to further special embodiments of the method according to the invention, the sequences coding for triacylglycerol lipase are homologs of calB_wt or calB_syn. calB from C. antarctica, especially calB_wt and/or calB_syn and homologs thereof, in particular homologs encoding functional equivalents, are, in each context described here, preferred representatives of nucleotide sequences encoding triacylglycerol lipases.

A homologous nucleotide sequence or a homologous nucleic acid or a homolog means, according to the invention, that not more than 40% of the nucleotides, in particular not more than 35% of the nucleotides, for example not more than 30% of the nucleotides or not more than 26%, 25%, 20%, 15%, 10%, 5%, 4%, 3%, 2% or 1% of the nucleotides are different when compared with a reference nucleotide sequence or reference nucleic acid. For example, a sequence homologous to SEQ ID NO:1 differs from SEQ ID NO:1 with respect to not more than 40% of the nucleotides, in particular not more than 35% of the nucleotides, for example not more than 30% of the nucleotides or not more than 26%, 25%, 20%, 15% or 10% of the nucleotides.

Homologous nucleotide sequences represent in particular sequences such as those that hybridize with the aforementioned reference nucleotide sequences under “stringent conditions”. This property is understood as the capacity of a poly- or oligonucleotide to bind under stringent conditions to an almost complementary sequence, whereas under these conditions nonspecific bonds between less complementary partners are absent. For this, the sequences should be complementary to 70-100%, preferably to 75%, 80%, 85% or 90% to 100%. The property of complementary sequences of being able to bind specifically to one another is utilized for example in Northern Blot or Southern Blot techniques or in primer binding in PCR or RT-PCR. Usually oligonucleotides starting from a length of 30 base pairs are used for this. “Stringent conditions” means, for example in the Northern Blot technique, the use of a hot washing solution at 50-70° C., preferably 60-65° C., for example 0.1×SSC buffer with 0.1% SDS (20×SSC: 3M NaCl, 0.3M Na-citrate, pH 7.0) for the elution of nonspecifically hybridized cDNA probes or oligonucleotides. As mentioned above, only highly complementary nucleic acids then remain bound to one another. The establishment of stringent conditions is known by a person skilled in the art and is described e.g. in Ausubel et al. (1989) (Sections 6.3.1-6.3.6). Homologous nucleic acids can be identified for example by examining genomic or cDNA banks and optionally can be amplified from them with suitable primers in PCR and then for example can be isolated with suitable probes.

Homologs of the triacylglycerol lipases according to the invention, in particular the lipases B according to the invention from Candida antarctica, can be identified by screening combinatorial banks of mutants, e.g. shortening mutants. For example, a bank of protein variants can be produced by combinatorial mutagenesis at the nucleic acid level, e.g. by enzymatic ligation of a mixture of synthetic oligonucleotides. There are a great many methods that can be used for the production of banks of potential homologs from a degenerated oligonucleotide sequence. The chemical synthesis of a degenerated gene sequence can be performed in an automatic DNA synthesizer, and the synthetic gene can then be ligated into a suitable expression vector. The use of a degenerated set of genes makes possible the provision of all sequences in a mixture, which encode the desired set of potential protein sequences. Methods for the synthesis of degenerated oligonucleotides are known by a person skilled in the art (e.g. Narang 1983; Itakura et al., 1984; Ike et al., 1983).

According to further embodiments, the nucleotide sequence encoding triacylglycerol lipase and in particular encoding CalB according to the invention is under the control of the T7 promoter, for example a promoter according to SEQ ID NO:3 or sequences homologous thereto. Suitable vectors, which allow expression under the control of the T7 promoter, are known by a person skilled in the art, for example the pET vector system (Novagen), e.g. the vectors pET-32a-c(+). According to the invention, calB_syn or calB_wt are prepared in pET-32b(+) (SEQ ID NO:7 or SEQ ID NO:8). Expression from these vectors takes place in particular at temperatures as defined above. According to the invention, it was found, surprisingly, that on expression of calB_wt or calB_syn from pET-vectors without using special cold-inducible promoters, an increased proportion of functional protein was formed by incubation at low temperatures.

According to further embodiments, the nucleotide sequence encoding triacylglycerol lipase and in particular encoding CalB according to the invention is under the control of a promoter that is inducible by cold shock. For the purposes of the present invention, cold shock means that the promoter is exposed to low temperatures. A suitable promoter that is inducible by cold shock is the promoter of the principal cold-shock gene cspA of E. coli (SEQ ID NO:4) (Goldstein et al., 1990). Expression vectors containing this promoter, which make it possible to clone a desired target gene by ordinary methods, are known by a person skilled in the art, for example the vectors pCOLD in their various forms from Takara Bio Inc., Japan (Takara 2003). According to the invention, calB_syn or calB_wt are prepared in pCOLDIII (SEQ ID NO:9 or SEQ ID NO:10). It was found, surprisingly, that on expression from these vectors in Origami™ 2(DE3) cells and Origami™ B cells, an increased amount of functional protein is formed. Induction of the cold-shock promoter takes place by incubation at low temperatures and optionally with addition of further factors necessary for expression (for example IPTG in the case of genes that are under the control of the lac-operator). Controlled establishment of low temperatures can optionally be facilitated by prior incubation (for example 30-minute incubation) on ice. Other cold-inducible promoters are known by a person skilled in the art, and are described for example in Qoronfleh et al., 1992, Nakashima et al. 1996 or Giladi at al. 1995.

Without being bound to a theory, it is assumed that a strategy for increased functional expression of triacylglycerol lipases, in particular CalB and its functional equivalents, consists of permitting expression of the protein essentially only at low temperatures. if expression takes place at temperatures above that, incorrectly folded protein may form, which acts as a crystallization nucleus, disturbing the formation of functional protein, even if expression takes place later under conditions that normally lead to the functional protein (for example low temperatures). Implementation of this strategy in accordance with the invention comprises expression under the control of promoters that only permit notable expression at low temperatures (for example the promoters contained in pCOLD vectors), with expression optionally being additionally controlled by transcription repressors (for example the gene product of lacI, which in the absence of IPTG prevents transcription and permits it if IPTG is present). Another implementation consists of using promoters, in particular strong promoters, whose transcription activity can be strictly controlled, and which permit transcription of these promoters only at low temperatures. As well as pET-vectors that comprise T7-promoters, other promoters or combinations of promoters and regulating elements (for example repressors) are known by a person skilled in the art, e.g. C1-regulated promoters (Schofield et al., 2002), the PItetO-1 promoter (Lutz and Bujard, 1997) or rhaT promoters (Giacalone et al. 2006).

The incubation time is selected for each vector system used so that a maximum amount of functional protein is formed, and can easily be determined by a person skilled in the art with routine tests for the protein that is to be expressed in each case from a given nucleic acid. The usual lengths of time are 1 to 48 hours, for example 8, 12, 16 and 24 hours.

According to another embodiment of the method according to the invention, simultaneously with the nucleotide sequence encoding triacylglycerol lipase and in particular that encoding CalB, one or more chaperones are expressed. The chaperones are for example selected from GroES, GroEL, DnaK, DnaJ, GrpE and Trigger Factor (TF) of E. coli. Expression is possible in any combinations, but in particular the combinations that are co-expressed are GroEL and GroES; or DnaK, DnaJ and GrpE; or DnaK, DnaJ, GrpE, GroES and GroEL; or Trigger Factor is co-expressed, optionally together with GroES and GroEL. The chaperones can be expressed jointly with the nucleotide sequence encoding the triacylglycerol-lipase from a vector. Alternatively the nucleotide sequence encoding triacylglycerol lipase and the nucleotide sequence(s) encoding chaperone(s) from separate vectors can be expressed. Suitable vectors are contained in the commercially available “Chaperone Plasmid Set”, which comprises the plasmids pG-KJE8, pGro7, pKJE7, pG-Tf2 and pTf16 (Takara Biomedicals, Japan, Takara 2003b).

Another object of the invention relates to a method for the detection of triacylglycerol lipases, wherein
i) a protein, for which triacylglycerol lipase activity is presumed, is expressed according to one of the aforementioned methods according to the invention,
ii) the expression product is contacted with a substrate that is hydrolyzable by triacylglycerol lipase, and
iii) the hydrolysis activity is determined.

This method is suitable for the identification, i.e. screening of new triacylglycerol lipases, in particular those with enzymatic activity comparable to that of lipase B from Candida antarctica. To a person skilled in the art it is also apparent that the method can be applied similarly for lipases that in their functional form require one or more S—S bridges, for example 1, 2, 3, 4, 5 or 6 S—S bridges per peptide chain, and the S—S bridges can be formed between sulfur-containing amino acids of the same peptide chain (intramolecular) and/or sulfur-containing amino acids of different peptide chains (intermolecular).

Suitable hydrolyzable substrates are known by a person skilled in the art, and demonstration of hydrolysis activity can also be carried out in the usual way. For example, tributyrin when added to agar plates at suitable concentrations (e.g. 1%) leads to their clouding, which disappears during enzymatic hydrolysis. Other suitable substrates are compounds whose hydrolytic cleavage leads to a color change, which can for example be detected photometrically (e.g. p-nitrophenyl palmitate). The hydrolysis activity can also be determined by enzymatic cleavage of carboxylic acid esters in the pH-stat assay. The lowering of the pH value caused by the carboxylic acids that are released is kept constant by titration with NaOH. The NaOH consumption, which is proportional to the amount of carboxylic acid released, therefore provides information on the hydrolysis activity of the enzyme. Regarding the aforementioned methods of detection, reference is made to Rusnak, 2004 (Chapter 8.4.2), which is taken fully into account by reference.

Other substrates hydrolyzable by triacylglycerol lipase can be determined by attempting to hydrolyze a given substrate with triacylglycerol lipases that are known by a person skilled in the art (for example CalB with the sequence CAA83122.1). If hydrolysis occurs, then the substrate can be used in the method according to the invention for the detection of triacylglycerol lipases.

Although the method of detection described above can be carried out in ordinary Petri dishes or cell culture vessels, the use of microtiter plates is envisaged in a special embodiment. For example, expression of the protein with presumed triacylglycerol lipase activity can already take place in the microtiter plate, for instance by cultivation of the prokaryotes in the microtiter plate and induction of expression of the protein. Furthermore, detection of hydrolysis activity can also take place in the microtiter plate, and depending on the parameters of the given culture, detection can take place without prior removal of the microorganisms. For example, at low cell densities in the respective wells of the microtiter plate, the hydrolysis activity can be determined directly from the change in turbidity properties of the medium or conversion of a photometric substrate, without the respective measured values being falsified by a high cell density. Alternatively, after expression of the protein with presumed triacylglycerol lipase activity released into the culture medium, the prokaryotic cells can be separated from the medium (for example by sedimentation of the cells by centrifugation and removal of the supernatant or immediate removal of the medium in the case of adherent cells growing on the surface of the well, or cells bound to a support) and the medium containing protein with presumed triacylglycerol lipase can be transferred to new microtiter plates for determination of hydrolysis activity.

According to a special embodiment, expression of the protein with presumed triacylglycerol lipase activity takes place in the presence of a hydrolyzable substrate, for example a substrate dissolved in a culture medium. According to another embodiment, prokaryotes that express a protein with presumed triacylglycerol lipase activity can be cultivated for example on a medium that contains a hydrolyzable substrate. Agar media that are cloudy owing to their content of tributyrin are particularly suitable in this connection. On expression of a hydrolytically active triacylglycerol lipase, for example after induction of the gene coding for triacylglycerol lipase by chemical substances or temperature change, depending on the expression vector used, there is cleavage of the tributyrin and hence clarification of the turbid agar. On areas of the agar with sufficient triacylglycerol lipase activity there is therefore formation of a halo, which can be detected visually or automatically with suitable image processing systems (e.g. Quantimet 500 Qwin; Leica, Cambridge, Great Britain) and can serve for identification of a bacterial colony that gives rise to triacylglycerol lipase activity. For a person skilled in the art, other modifications of the method will be apparent, for example inoculation of tributyrin-agar-filled wells of microtiter plates with bacterial suspensions at dilutions that allow individual bacterial colonies to develop in each well, and subsequent visual or automated image analysis.

According to another embodiment, the expressed protein with presumed triacylglycerol lipase activity is separated from the expressing prokaryotic cells, in particular E. coli, and then contacted, in the cell-free state, with the hydrolyzable substrate. Suitable methods of separation are known by a person skilled in the art, for example sedimentation of the cells by centrifugation or separation by filtration. This embodiment has the advantage that the subsequent detection of hydrolysis activity is not affected by the presence of the prokaryotic cells.

According to a special embodiment, said detection takes place photometrically by determination of the decrease in hydrolyzable substrate or increase in hydrolysis product. Suitable substrates are known by a person skilled in the art, for example p-nitrophenyl esters such as p-nitrophenyl palmitate or p-nitrophenyl acetate. The parameters of the photometric measurement can be readily adapted by a person skilled in the art to the substrates and solutions used (for example, when using p-nitrophenyl palmitate (pNPP) it is recommended to measure the increase in extinction at 410 nm).

In a special embodiment, the method of detection according to the invention is used for the screening of mutagenized proteins or proteins encoded by mutagenized nucleic acids for triacylglycerol lipase activity. These can be proteins that are to be endowed with triacylglycerol lipase activity by mutagenesis (for example by incorporating sequence motifs that are recognized as being important for this enzymatic activity into proteins that have little if any triacylglycerol lipase activity), or proteins with already known triacylglycerol lipase activity, which is to be modified by introducing one or more mutations. Mutagenesis (which leads to a mutation or to a mutated nucleotide sequence or a mutated protein) means, with reference to nucleotide sequences, the adding, removing or exchanging of at least one nucleotide. With reference to proteins it means the adding, removing or exchanging of at least one amino acid. A mutagenized protein according to the invention is in particular a protein whose coding nucleotide sequence comprises the nucleotide sequence encoding lipase B from Candida antarctica (CalB), or a protein whose coding nucleotide sequence has at least one mutation relative to the nucleotide sequence encoding a lipase B from Candida antarctica (CalB), or a protein whose coding nucleotide sequence is homologous to the coding nucleotide sequence of a lipase B from Candida antarctica (CalB), or a protein that is a functional equivalent to a lipase B from Candida antarctica (CalB). For all these proteins, a mutagenization of the nucleotide sequence encoding the respective protein need not necessarily lead to a change in the amino acid sequence of the expressed protein. As already mentioned above, methods are known by a person skilled in the art for optimizing the nucleotide sequence of genes for example with respect to the preferred codon usage in the host organism provided for expression, the avoidance of mRNA secondary structures or particular sequence motifs (e.g. the GeneOptimizer™ technology from GeneArt, Regensburg, Germany). These optimizations can serve for improving expression both at the transcription level and at the translation level and at the same time, utilizing the degenerated code that permits several base triplets for certain amino acids, serve for the translation of an unaltered protein. In this case the method of detection according to the invention serves for monitoring the expression of a functional protein. On the other hand, by mutagenesis of coding nucleotide sequences, proteins can be produced whose amino acid sequence is different, compared with proteins that are expressed by nonmutagenized starting sequences. In this case, with the method of detection according to the invention it is possible to verify whether the proteins encoded by the mutagenized nucleotide sequences have, in comparison with the proteins encoded by the nonmutagenized starting sequences, unchanged hydrolysis activity or modified hydrolysis activity, for example that is decreased (or absent), increased, or altered with respect to one or more parameters of enzymatic activity. As said parameters, which in particular can be tested by selection of the conditions in which a mutagenized protein with presumed triacylglycerol lipase activity according to step ii) of the method of detection according to the invention is contacted with a hydrolyzable substrate, consideration may be given for example to substrate specificity, enantioselectivity or turnover number of the protein and its dependence for example on temperature, pH, ion concentration and/or presence of possible inhibitors or activators.

Mutagenesis by targeted changes of the nucleotide sequence, for example by means of the polymerase chain reaction (PCR), and by undirected changes, for example by chemical mutagenesis, is known by a person skilled in the art and is described for example in Roufa 1996 or Kirchhoff and Desrosiers 1996.

The use described above of the method of detection according to the invention is suitable in particular for the screening of gene banks. For example, a bank of protein variants can be produced by combinatorial mutagenesis at the nucleic acid level, e.g. by enzymatic ligation of a mixture of synthetic oligonucleotides. There is a large number of methods that can be used for the production of banks of potential homologs from a degenerated oligonucleotide sequence. The chemical synthesis of a degenerated gene sequence can be carried out in an automatic DNA synthesizer, and the synthetic gene can then be ligated into a suitable expression vector. The use of a degenerated set of genes makes it possible to provide all sequences in a mixture that encode the desired set of potential protein sequences. Methods of synthesis of degenerated oligonucleotides are known by a person skilled in the art (e.g. Narang 1983; Itakura at al., 1984); Itakura at al. 1984; Ike et al. 1983).

The use according to the invention is suitable in particular for the high-throughput screening of samples, for example the screening of a large number of samples one after another in a short time or the simultaneous screening of several parallel samples or a combination thereof. Thus, in particular it is a suitable method for the screening of the gene banks described above. For example, these can be screened for clones that display an especially high functional expression of triacylglycerol lipases, or those that express triacylglycerol lipases with altered properties. In this connection, in particular the use of microtiter plates is advantageous for the expression of one or more proteins under investigation and/or for the determination of its/their hydrolysis activity. As is known by a person skilled in the art, a high sample throughput can be achieved by automation, for example with pipetting robots, which transfer supernatants containing protein with presumed hydrolase activity from the wells of the microtiter plates used for expression of these proteins into wells intended for the detection of hydrolysis activity, or pipette detection reagents into the wells containing said supernatants. Furthermore, photometric assessment using microtiter plates can be carried out in particular with plate readers, which automatically measure the extinction of the solutions contained in the individual wells. When using agar plates, clear haloes arising through hydrolysis activity or haloes optically detectable in other ways, as already described above, can be detected for example using suitable image processing systems (e.g. Quantimet 500 Qwin; Leica, Cambridge, Great Britain).

Another object of the invention relates to a method of production of a triacylglycerol lipase (E.C. 3.1.1.3), in which an expressed protein with activity of a triacylglycerol lipase (E.C. 3.1.1.3) is detected by one of the methods of detection described above, the strain expressing this protein is cultivated under lipase-expressing conditions and optionally the expressed lipase is isolated. Suitable methods of isolation, which can if necessary be adapted by routine tests for the particular protein, are known by a person skilled in the art and for example are described below. It will be apparent to a person skilled in the art that the method can be applied similarly for lipases that in their functional form require one or more S—S bridges, for example 1, 2, 3, 4, 5 or 6 S—S bridges per peptide chain, and the S—S bridges can be formed between sulfur-containing amino acids of the same peptide chain (intramolecular) and/or sulfur-containing amino acids of different peptide chains (intermolecular).

Another object of the invention relates to triacylglycerol lipases that can be obtained by the methods of production described above, for example triacylglycerol lipases occurring in the cellular environment of the prokaryotic host or partially, largely or completely purified triacylglycerol lipases. Especially suitable triacylglycerol lipases are lipases B from Candida antarctica, which differ from the sequence deposited under access number CAA83122.1 at GenBank by at least one amino acid, and proteins homologous to the sequence deposited under GenBank access number CAA83122.1, which represent functional equivalents.

“Functional equivalents” means in particular, according to the invention, mutants that differ in at least one sequence position from the amino acid sequence of a triacylglycerol lipase taken as a basis, in particular any lipase B, but nevertheless possess one of the aforementioned biological activities, for example constant, decreased or increased hydrolysis activity, or altered substrate specificity, enantioselectivity or turnover number and their dependence for example on temperature, pH, ion concentration, or presence of possible inhibitors or activators.

“Functional equivalents” therefore comprise mutants obtainable by one or more amino acid additions, substitutions, deletions and/or inversions, with said changes occurring in any sequence position, provided they lead to a mutant with the profile of properties according to the invention. Functional equivalence is in particular also present if the reactivity patterns between mutant and unchanged polypeptide coincide qualitatively, i.e. for example the same substrates are converted at a different rate.

“Precursors” of the polypeptides described and “functional derivatives” and “salts” of the polypeptides are also “functional equivalents” in the above sense.

“Precursors” are natural or synthetic preliminary stages of the polypeptides with or without the desired biological activity.

The expression “salts” means in this context both salts of carboxyl groups and salts of acid addition of amino groups of the protein molecules according to the invention. Salts of carboxyl groups can be produced by well-known methods and comprise inorganic salts, such as sodium, calcium, ammonium, iron and zinc salts, and salts with organic bases, for example amines, such as triethanolamine, arginine, lysine, piperidine and the like. Salts of acid addition, for example salts with mineral acids, such as hydrochloric acid or sulfuric acid and salts with organic acids, such as acetic acid and oxalic acid are also objects of the invention.

“Functional derivatives” of enzymes according to the invention can also be produced on functional amino acid side groups or at their N- or C-terminal end by known techniques. Said derivatives comprise for example aliphatic esters of carboxylic acid groups, amides of carboxylic acid groups, obtainable by reaction with ammonia or with a primary or secondary amine; N-acyl derivatives of free amino groups, produced by reaction with acyl groups; or O-acyl derivatives of free hydroxyl groups, produced by reaction with acyl groups.

“Functional equivalents” naturally also comprise polypeptides that can be obtained from other organisms, and naturally occurring variants. For example, homologous sequence regions can be established by sequence comparison and equivalent enzymes can be determined on the basis of the concrete specifications of the invention.

“Functional equivalents” also comprise fragments, in particular individual domains or sequence motifs, of the polypeptides according to the invention, which for example have the desired biological activity.

“Functional equivalents” are, in addition, fusion proteins that have one of the natural racemase sequences or functional equivalents derived therefrom and at least one other, functionally different, heterologous sequence in functional N- or C-terminal linkage (i.e. without substantial mutual functional impairment of the fusion protein moieties). Nonlimiting examples of such heterologous sequences are e.g. signal peptides or enzymes.

“Functional equivalents” covered by the invention are proteins that are homologous to the natural proteins. They possess at least 60%, preferably at least 75%, in particular at least 85%, for example at least 90%, 95% or 99%, homology to one of the natural amino acid sequences, calculated according to the algorithm of Pearson and Lipman (Pearson and Lipman 1988). A percentage homology of a homologous polypeptide according to the invention means in particular percentage identity of the amino acid residues based on the total length of one of the amino acid sequences of an enzyme according to the invention or an enzyme subunit. The present invention comprises in particular functional equivalents according to the definitions given above, which additionally have homology of at least 60%, preferably at least 75%, in particular at least 85%, for example at least 90%, 95% or 99%, to the starting sequence.

In the case of a possible protein glycosylation, “functional equivalents” according to the invention comprise proteins of the type designated above in deglycosylated or glycosylated form and modified forms that can be obtained by altering the glycosylation pattern.

Functional equivalents can be determined using the methods according to the invention. For example, proteins whose functional equivalence to triacylglycerol lipases and in particular CalB are to be determined, can be expressed by means of the method of expression according to the invention and investigated by the method of detection according to the invention. Expressed proteins that have hydrolysis activity, in particular altered hydrolysis activity in comparison with triacylglycerol lipase or CalB, are functional equivalents.

In this connection, prokaryotes expressing triacylglycerol lipase according to the invention can be cultivated and fermented by known methods. Then, if the polypeptides are not secreted into the culture medium, the cells are disrupted and the product is obtained from the lysate by known methods of protein isolation. The cells can be disrupted optionally by high-frequency ultrasound, by high pressure, e.g. in a French pressure cell, by osmolysis, by the action of detergents, lytic enzymes or organic solvents, using homogenizers or by a combination of several of the methods listed.

The polypeptides can be purified by known chromatographic methods, such as molecular sieve chromatography (gel filtration), such as Q-sepharose chromatography, ion exchange chromatography, affinity chromatography and hydrophobic chromatography, and by other usual methods such as ultrafiltration, crystallization, salting-out, dialysis and native gel electrophoresis. Suitable methods are described for example in Cooper (1980) or in Scopes (1981).

Another object of the invention relates to a nucleic acid coding for triacylglycerol lipase, in particular a nucleic acid coding for lipase B from Candida antarctica, which comprises a coding nucleotide sequence according to SEQ ID NO:1, or comprises a nucleotide sequence that differs from SEQ ID NO:2 by at least one nucleotide, or comprises a nucleotide sequence homologous thereto. The aforementioned nucleotide sequences encode for example a functional equivalent of CalB. According to a special embodiment the nucleic acid comprises a coding nucleotide sequence according to SEQ ID NO:1 or a nucleotide sequence homologous thereto.

Another object of the invention relates to a recombinant vector that comprises a nucleic acid coding for a triacylglycerol lipase, which is operatively linked to at least one regulating nucleic acid sequence. According to another special embodiment the nucleic acid sequence encoding triacylglycerol lipase comprises a nucleotide sequence according to SEQ ID NO:1 or SEQ ID NO:2. “Operative linkage” means the sequential arrangement of promoter, coding sequence, terminator and optionally other regulating elements in such a way that each of the regulating elements can fulfill its function in expression of the coding sequence as defined. Examples of operatively linkable sequences are targeting sequences and enhancers, polyadenylation signals and the like. Other regulating elements comprise selectable markers, amplification signals, replication origins and the like. Suitable regulatory sequences are described for example in Goeddel 1990. According to the invention, it was found for example that in particular, on co-expression with pGRO7, functional expression of calB_syn (SEQ ID NO:1) and calB_wt (SEQ ID NO:2) was increased.

As well as plasmids and phages, “vectors” also means all other vectors known by a person skilled in the art, thus e.g. viruses, such as SV40, CMV, baculovirus and adenovirus, transposons, IS elements, phasmids, cosmids, and linear or circular DNA. These vectors can be replicated autonomously in the host organism or can be replicated chromosomally. These vectors represent a further embodiment of the invention. Suitable plasmids are for example pLG338, pACYC184, pBR322, pUC19, pKC30, pRep4, pHS1, pKK223-3, pDHE19.2, pHS2, pPLc236, pMBL24, pLG200, pUR290, pIN-III¹¹³-B1, Igt11 or pBdCl in E. coli, pIJ101, pIJ364, pIJ702 or pIJ361 in Streptomyces, pUB110, pC194 or pBD214 in Bacillus, pSA77 or pAJ667 in Corynebacterium. The aforementioned plasmids represent a small selection of the possible plasmids. Other plasmids are certainly known by a person skilled in the art and can for example be found in the book Cloning Vectors (publ. Pouwels P. H. et al. Elsevier, Amsterdam-New York-Oxford, 1985, ISBN 0 444 904018). Suitable vectors are those that permit the functional expression of the nucleotide sequence coding for a protein with triacylglycerol lipase activity, which can in turn be determined by the method of detection described above.

Special embodiments comprise the vector pET32b(+) and the other representatives of the pET vector family (Novagen 1999), in particular pET-32a-c, pET-41a-c and pET-42a-c, and pCOLD III and other representatives of the pCOLD vector family (e.g. pCOLD I, pCOLD II, pCOLD IV, pCOLD TF, obtainable for example from Takara Bio Europe S.A.S, Gennevilliers, France). According to quite particular embodiments, pET-32b(+)/calB_syn (SEQ ID NO:7, calB_syn in pET-32b(+)), pET-32b(÷)/calB_wt (SEQ ID NO:8, calB_wt in pET-32b(+)), pCOLDIII/calB_syn (SEQ ID NO:9, calB_syn in pCOLDIII) and pCOLDIII/calB_wt (SEQ ID NO:10, calB_wt in pCOLDIII) are prepared according to the invention as recombinant vectors that comprise a nucleic acid coding for a triacylglycerol lipase.

Another object of the invention relates to a recombinant host cell that comprises such a vector or a nucleic acid coding for triacylglycerol lipase, which comprises a coding nucleotide sequence according to SEQ ID NO:1 or a nucleotide sequence homologous thereto. The recombinant cell can be in particular a prokaryotic cell, in particular an E. coli cell. Further examples of prokaryotic cells are, among the Gram-negative bacteria, representatives of the Enterobacteriaceae such as Salmonella, Shigella, Serratia, Proteus, Klebsiella or Enterobacter, Pseudomonas; among the Gram-positive bacteria for example representatives of the genus Bacillus, e.g. B. subtilis and B. licheniformis.

Further objects of the invention relate to the use of coding nucleic acid sequence according to the invention, a vector according to the invention or a host cell according to the invention for carrying out a method according to the invention for the expression of a functional triacylglycerol lipase, a method according to the invention for the detection of a functional triacylglycerol lipase, or a method according to the invention for the production of a functional triacylglycerol lipase. It was found that when the vectors according to the invention were used in a method of expression according to the invention, in particular at low incubation temperatures, calB_wt and calB_syn were surprisingly expressed functionally to an increased extent in pET-32b(+) (SEQ ID NO: 8 or SEQ ID NO:7) or in pCOLDIII (SEQ ID NO:10 or SEQ ID NO:9). Moreover, it was found according to the invention that on co-expression with pG-KJE8 and in particular co-expression with pGRO7, pG-Tf2, or pTf16, calB_syn (SEQ ID NO:1) and calB_wt (SEQ ID NO:2) were functionally expressed to a particularly high degree.

It will be apparent to a person skilled in the art that the method according to the invention for the expression of functional triacylglycerol lipases, the method for their detection and the method of production thereof can also be applied analogously to enzymes other than triacylglycerol lipases. For example, these methods can be adapted to enzymes of EC Class 3.1 (ester-hydrolases) or generally to those of EC Class 3 (hydrolases) (http://www.iubmb.unibe.ch; http://www.chem.qmul.ac.uk/iubmb/enzyme/). The methods according to the invention accordingly extend to all enzymes that can be expressed, detected or produced using the general principles disclosed in these methods, in particular those enzymes for whose functionality the formation of one or more S—S bridges, for example 1, 2, 3, 4, 5 or 6 S—S bridges, is necessary, wherein the S—S bridges can be formed between sulfur-containing amino acids of the same peptide chain (intramolecular) and/or sulfur-containing amino acids of different peptide chains (intermolecular).

### Examples

I. General Information

Chemicals

Unless stated otherwise, all chemicals were obtained from Fluka (Buchs, Switzerland), Sigma-Aldrich (Taufkirchen, Germany) and Roth GmbH (Karlsruhe, Germany).

Strains and Plasmids

E. coli DH5α was obtained from Clontech (Heidelberg, Germany). E. coli Origami™ 2(DE3), Origami™ B and the plasmid pET-32b(+) were obtained from Novagen (Darmstadt, Germany). The plasmid pUC18 was obtained from MBI Fermentas (St. Leon-Rot, Germany), pColdIII and the chaperone-plasmid set, which contains the plasmids pG-KJE8, pGro7, pKJe7, pG-Tf2 and pTf16, were obtained from Takara (Otsu, Japan). The construct pPCR/CalB, which contained the codon-optimized calB gene, was synthesized by GENEART (Regensburg, Germany). The construct pPICZαA/calB was described earlier (Rusnak 2004).

Cloning of calB Variants

calB_wt was amplified from the template construct pPICZαA/calB using the primers wt_pUC18_fw and wt_pUC18_rev (Table 1) and then cloned into the vector pUC18, obtaining the construct pUC18/calB_wt. For the subcloning in pET-32b(+), the lipase gene was amplified using the primers wt_pET-32b(+)_fw and wt_pET32b(+)_rev (Table 1, see below) and then cloned into the vector via the EcoR1 and NotI restriction sites (pET-32b(+)/calB_wt). For the subcloning in pColdIII, CalB was amplified using the primers wt_pColdIII_fw and wt_pColdIII_rev (Table 1) and then cloned into the vector using standard methods (pColdIII/CalB_wt).

CalB_syn was amplified from pPCR/calB using the primers syn_pUC18/pET-32b(+)_fw and syn_pUC18_rev, syn_pUC18/pET-32b(+)_fw and syn_pET-32b(+)_rev or syn_pColdIII_fw and syn-pColdIII_rev (Table 1) and then cloned into the plasmids pUC18/calB_syn, pET-32b(+)/calB_syn and pColdIII/calB_syn, using standard methods, obtaining the constructs pUC18/calB_syn, pET-32b(+)/calB_syn and pColdIII/calB_syn.

The following Table 1 shows the primers used for the subcloning of calB variants. The restriction sites are underlined.

TABLE 1

| Primer | Sequence | Restriction site |
|---|---|---|
| wt-pUC18_fw | gatgaattcgctaccttccggttcggacc | EcoR1 |
| wt-pUC18_rev | ccacatatgtcagggggtgacgatgcc | NdeI |
| wt_pET-32b(+)_fw | ccggaattcgctaccttccggttc | EcoR1 |
| wt_pET-32b(+)_rev | cggcatcgtcaccccctaagcggccgc | NotI |
| wt_pColdIII_fw | cgattcatatgctaccttccggttcggacc | NdeI |
| wt_pColdIII_rev | ccttaagaattctcagggggtgacgatgcc | EcoR1 |
| syn_pUC18/pET-32b(+)_fw | ccggaattcgctgccgagcgg | EcoR1 |
| syn_pUC18_rev | gtattgtgaccccgtaataagcatatggaattcc | NdeI |
| syn_pET-32b(+)_rev | gcggtattgtgaccccgtaagcttggg | HindIII |
| syn_pColdIII_fw | cagttcatatgctgccgagcggtagcgat | NdeI |
| syn_pColdIII_rev | ccttaagaattcttacggggtcacaataccgct | EcoR1 |

Lipase Expression and Co-Expression of Chaperones

Expression experiments were repeated four to six times and activities are stated as mean values.

pUC18 Expression:

Origami™ B and DH5α cells were transformed with pUC18 constructs. The cells were grown to an optical density of 0.6 at 600 nm at 37° C. and 180 rev/min in 100 ml LB medium (Luria 1960), which contained 100 μg/ml ampicillin (LBₐₘₚ). Then lipase expression was induced by addition of IPTG (final concentration 1 mM). The cells were grown for an additional 4 hours at 30° C. and 180 rev/min and were harvested by centrifugation at 4000 g for 30 min at 4° C.

pET-32b(+)-Expression:

Origami™ 2(DE3) cells were transformed with pET32b(+) constructs. The cells were grown at 37° C. and 180 rev/min to an optical density of 0.6 at 600 nm in 100 ml LBₐₘₚ and processed as described previously. Alternatively the cells were cooled on ice before induction and expression was carried out for 24 hours at 15° C.

pColdIII-Expression:

Origami™ B cells were transformed with pColdIII constructs. The cells were grown at 37° C. and 180 rev/min to an optical density of 0.4-0.6 at 600 nm in 100 ml LBₐₘₚ. Then the cultures were cooled on ice for 30 min and lipase expression was induced by adding IPTG (final concentration 1 mM). The cells were grown for a further 24 hours at 15° C. and 180 rev/min and were harvested by centrifugation at 4000 g for 30 min at 4° C.

Co-Expression of Chaperone Plasmids and pColdIII Constructs:

Origami™ B cells were transformed with chaperone plasmids. The cells were grown at 37° C. in 100 ml LB, which contained 34 μg/ml chloramphenicol, and competent cells were produced by the usual methods. The recombinant cells were transformed with the pColdIII constructs and selected on LB_{cm+amp}. For expression, the cells were grown at 37° C. and 180 rev/min to an optical density of 0.4-0.6 at 600 nm in LB_{cm+amp}, which (in the case of pGro7, pKJE7 and pTf16) contained 1 mg/ml L-arabinose and (in the case of pG-Tf2) 5 ng/ml tetracycline or (in the case of pG-KJE8) L-arabinose and tetracycline at the concentrations stated above. The cultures were cooled on ice for 30 min. Then lipase expression was induced by adding IPTG (final concentration 1 mM). The cells were grown for a further 24 hours at 15° C. and 180 rev/min and were harvested by centrifugation at 4000 g for 30 min at 4° C.

Tributyrin-Agar Plate Assay

Cells were cultivated on LB-agar plates that contained 1% emulsified tributyrin and the corresponding antibiotics and 1 mg/ml L-arabinose in co-expression of pGro7, pKJE7 or pTf16, 5 ng/ml tetracycline in co-expression of pG-Tf2, or L-arabinose and tetracycline in co-expression of pG-KJE8. After growing the cells for 24 h at 37° C., the plates were covered with a layer of soft agar (0.6% agar in water) that contained 1 mM IPTG, and for expression were incubated at 30° C., 15° C. or room temperature. The expression of functional lipase was indicated by the formation of clear haloes around the colonies.

Lipase Activity Assay, SDS-PAGE and Densitometric Analysis

The cells were disrupted by ultrasonic treatment three times for a duration of 30 s each time, in 50 mM sodium phosphate buffer (pH 7.5), and cell debris was removed by centrifugation. The cellular lysates were tested for activity using the pH Stat device (Metrohm, Filderstadt, Germany). The hydrolysis of the substrate (5% tributyrin, emulsified in water with 2% gum arabic) was monitored by titration with 10 mM NaOH. The protein content of the cell lysate was measured by the Bradford assay (Bradford 1976). One lipase activity unit was defined as release of 1 μmol fatty acid per minute. Insoluble and soluble fractions were investigated by SDS-PAGE according to the standard method (Laemmli 1970), using 50 μg of the clarified cell lysate (corresponding to 0.2-0.4 ml cell culture) or insoluble cell debris from 0.5 ml cell culture. The gels were stained with Coomassie Brilliant Blue. The percentage of soluble CalB relative to total cell protein was determined by densitometry using the “Scion Image” program (Frederick, Md., USA).

High-Throughput Expression and Activity Assay

pColdIII constructs and Origami™ B cells bearing the chaperone plasmid were grown to an optical density of 0.4-0.6 at 37° C. and 400 rev/min in a 96-well microtiter plate (Greiner, Nürtingen, Germany), which contained 150 μl LB_{cm+amp} plus chaperone inducer (see above). The cells were cooled on ice for 30 min and lipase expression was induced by adding IPTG to a final concentration of 1 mM. After expression for 24 h at 15° C. and shaking at 200 rev/min, the cells were harvested by centrifugation and lysed by adding 50 μl lysis buffer (50 mM sodium phosphate pH 7.5, 1 mg/ml lysozyme, 1 μl/DNAse). The lysates were incubated at 37° C. with shaking (300 rev/min) and cooled on ice for 30 min. After incubation at −80° C. for 1 hour, the cells were thawed at room temperature (RT) and cell debris was removed by centrifugation at 4000 rev/min for 30 min at 4° C.

To detect lipase activity, 20 μl clarified cell lysate was added to 180 μl assay solution (162 μl solution B (1 g Triton X-100+0.2 g gum arabic in 200 ml 0.1 M Tris-HCl pH 7.5)+18 μl solution A (60 mg pNPP in 20 ml n-propanol). The formation of p-nitrophenolate was measured after 5 min by measuring the extinction at 410 nm (Spectra Max 340PC, Molecular Devices Corp., Sunnyvale, Calif., USA). Alternatively, the lipase activity was quantified by means of the pH Stat assay (as described above).

II. Examples of Application

1. Cloning of calB from Candida antarctica

The calB_wt gene without the nucleotide sequence coding for the N-terminal pre-pro peptide sequence was cloned into the E. coli expression vectors pUC18, pET-32b(+) or pColdIII (Table 2). The pPICZαA/calB construct (Rusnak 2004), which contained the calB gene from Candida antarctica, served as template for amplification of the lipase gene.

The calB gene was optimized using the GeneOptimizer™ technology. In addition to optimization of codon preference, regions with very high (>80%) or low (<30%) GC content were avoided if possible. Furthermore, cis-acting sequence motifs such as internal TATA boxes, Chi sites, ribosomal linking sites, ARE, INS and CRS sequence elements as well as repetitive sequences and RNA secondary structures were avoided. The optimized gene (calB_syn) differs in 253 nucleotides (26.5%) from the calB_wt sequence; the changes that were effected are shown in FIG. 1. At the amino acid level, the synthetic gene encodes the published protein (CAA83122.1). Then the synthetic gene was subcloned into the expression vectors stated in Table 2.

TABLE 2
Table 2: Plasmids and strains used.

| | Gene of | | | | Resistance | |
|---|---|---|---|---|---|---|
| Plasmid | interest | Pro | Inducer | Ori | marker | Reference |
| pPICZαA/calB | calB_wt | AOX1 | Methanol | pUC | Zeozin ™ | (Invitrogen 2002) |
| pPCR/calB | calB_syn | / | / | ColE1 | Ampicillin | Geneart (Regensburg, |
| | | | | | | Germany) |
| pUC18/calB | calB_wt/calB_syn | lac | IPTG | pBR322 | Ampicillin | MBI Fermentas (St. |
| | | | | | | Leon-Rot, Germany) |
| pColdIII/calB | calB_wt/calB_syn | cspA | Cold shock + | ColE1 | Ampicillin | (TaKaRa 2003) |
| | | | IPTG | | | |
| pET32-b(+)/calB | Trx-calB_wt/Trx- | T7 | IPTG | pBR322 | Ampicillin | (Novagen 1998) |
| | calB_syn | | | | | |
| pGro7 | groES-groEL | araB | L-arabinose | pACYC | Chloramphenicol | (TaKaRa 2003) |
| pG-Tf2 | groES-groEL-tig | Pzt1 | Tetracycline | pACYC | Chloramphenicol | (TaKaRa 2003) |
| pTf16 | tig | araB | L-arabinose | pACYC | Chloramphenicol | (TaKaRa 2003) |
| pKJE7 | dnaK-dnaJ-grpE | araB | L-arabinose | pACYC | Chloramphenicol | (TaKaRa 2003) |
| pG-KJE8 | dnaK-dnaJ-grpE | araB | L-arabinose | pACYC | Chloramphenicol | (TaKaRa 2003) |
| | GroES-groEL | Pzt1 | Tetracycline | | | |

| Strains | Genotype | Reference |
|---|---|---|
| DH5α | supE44 ΔlacU169(Φ80lacZΔM15) hsdR17 recA1 end A1 gyrA96 | Clontech (Heidelberg, |
| | thi1relA1 | Germany) |
| Origami ™ B | Δara-leu7697 ΔlacX74 ΔphoAPvulI phoR araD139 ahpC galE galK | (Novagen 2004) |
| | rpsL F′[lac⁺(lacI^{q})pro] gor522::Tn10 (Tc^{R}) trxB::kan | |
| Origami ™ 2(DE3) | Δ(ara-leu)7697 ΔlacX74 ΔphoA pvulI phoR araD139 ahpC galE galK | (Novagen 2004) |
| | rpsL F′[lac+ lacI q pro] (DE3) gor522::Tn10 trxB (StrR, TetR) | |

Pro: promoter, Ori: replication origin

2. Lipase Expression in Three Different Vector Systems

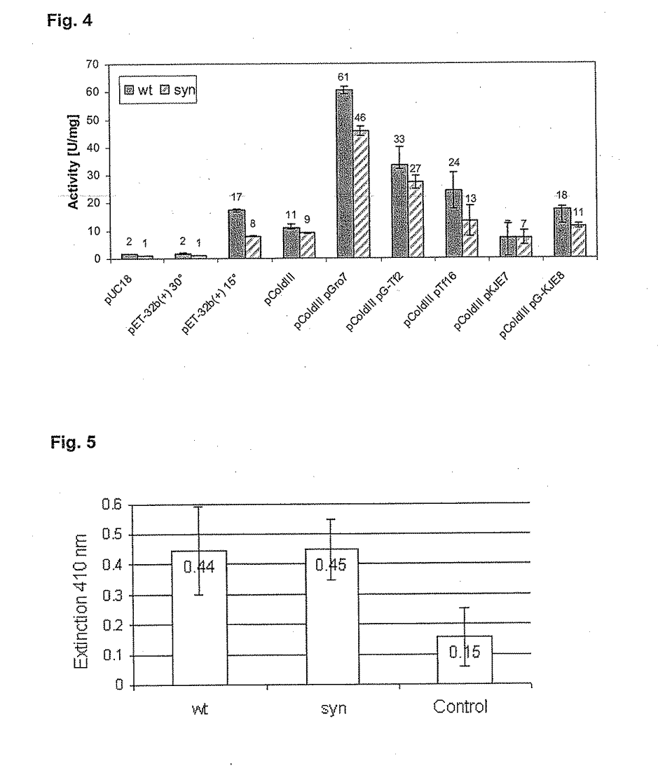

For the expression of calB-encoding vectors, the strains E. coli Origami™ B and Origami™ 2(DE3) were used, which are characterized by their thioredoxin reductase and glutathione reductase deficiency. pUC18/calB_wt-transformed Origami™ B cells showed halo formation on tributyrin-agar plates, whereas this was not so for the comparative strain DH5α (data not shown). However, the CalB activity was very low in the Origami™ B cells transformed with pUC18/calB_wt or pUC18/calB_syn and corresponded for both constructs to hydrolysis of only about 2 U tributyrin per milligram of total soluble protein (FIG. 4). 1 U (unit) is defined as the turnover of 1 μmol substrate per minute. In an SDS-PAGE analysis, no protein band was detected in the soluble fraction, which corresponded to the mass of CalB (33 kD) (data not shown), whereas the CalB content of the insoluble fraction was 10-12% (Table 3).

TABLE 3
Table 3: Densitometric analysis of the CalB content in
cellular extracts from various expression experiments.

| | CalB content of | | CalB content of | | CalB content of | | Content of soluble | |
|---|---|---|---|---|---|---|---|---|
| | insoluble fraction | | soluble fraction | | the total cell | | CalB in the total | |
| | [%] | | [%] | | protein [%] | | cell protein [%] | |
| | wt | syn | wt | syn | wt | syn | wt | syn |
| pUC18 | 12 | 10 | n.d. | n.d. | 4 | 3 | n.d. | n.d. |
| pET32-b(+) (30° C.) | 19 | 18 | n.d. | n.d. | 6 | 6 | n.d. | n.d. |
| pET32-b(+) (15° C.) | 24 | 26 | n.d. | n.d. | 8 | 9 | n.d. | n.d. |
| pColdIII | 42 | 38 | n.d. | n.d. | 14 | 13 | n.d. | n.d. |
| pColdIII + pGro7 | 49 | 28 | 11 | 10 | 23 | 16 | 7 | 7 |
| pColdIII + pG-Tf2 | 42 | 29 | 9 | 7 | 20 | 14 | 6 | 5 |
| pColdIII + pTf16 | 22 | 19 | 10 | 5 | 14 | 9 | 7 | 3 |
| pColdIII + pKJE7 | 31 | 15 | n.d. | n.d. | 10 | 5 | n.d. | n.d. |
| pColdIII + pG-KJE8 | 5 | 6 | 4 | 4 | 4 | 5 | 3 | 3 |

The SDS-PAGE gels stained with Coomassie Blue were evaluated using the Scion Image program, n.d.: not detectable.

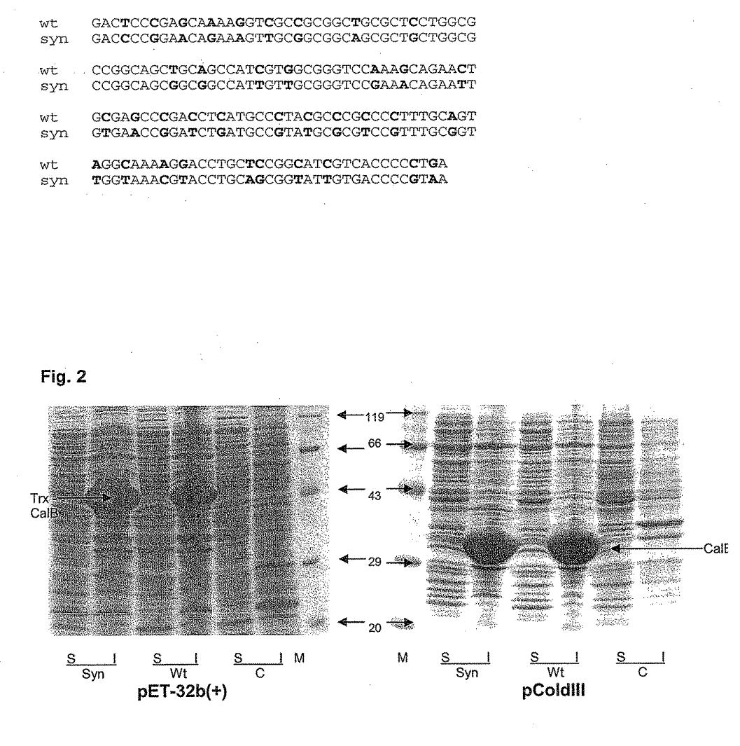

To increase the yield of active enzyme, the genes calB_wt and calB_syn were fused using the vector pET-32b(+) with a thioredoxin-tag (Trx•TAG™) and expressed in E. coli Origami™ 2(DE3). On incubation at room temperature the transformed cells showed clear halo formation, whereas cultivation at 37° C. did not lead to any detectable enzymatic activity. Whereas the expression of Trx-CalB in shaken-flask culture at 30° C. led to a marked increase in the amount of enzyme in the insoluble fraction (18-19%, Table 3), but not to an increase in solubility and hence in activity of CalB, expression at 15° C. produced up to 17 U/mg soluble protein with the wt-gene (calB_wt) and 8 U/mg with the synthetic gene (calB_syn) (FIG. 4). In this system, moreover, halo formation on tributyrin-agar plates was only observed at low cultivation temperatures. Nevertheless, using SDS-PAGE, a large amount of insoluble protein was detected on expression from pET-32b(+) (24% and 26%) and from pColdIII (42% and 38%), even with expression at 15° C. (FIG. 2, Table 3).

3. Lipase Expression with Co-Expression of Molecular Chaperones

The pColdIII constructs according to example of application 2 were co-expressed with several combinations of chaperones that are supplied in the TaKaRa Chaperone Plasmid Set (see Table 4 below).

TABLE 4
Table 4: Constituents of the Chaperone Plasmid Set from TaKaRa

| | | | | Resistance |
|---|---|---|---|---|
| Plasmid | Chaperone | Promoter | Inducer | marker |
| pG-KJE8 | dnaK-dnaJ-grpE | araB | L-Arabinose | Cm |
| | GroES-groEL | Pzt1 | Tetracycline | |
| pGro7 | groES-groEL | araB | L-Arabinose | Cm |
| pKJE7 | dnaK-dnaJ-grpE | araB | L-Arabinose | Cm |
| pG-Tf2 | groES-groEL | Pzt 1 | Tetracycline | Cm |
| pTf16 | Tig | araB | L-Arabinose | Cm |

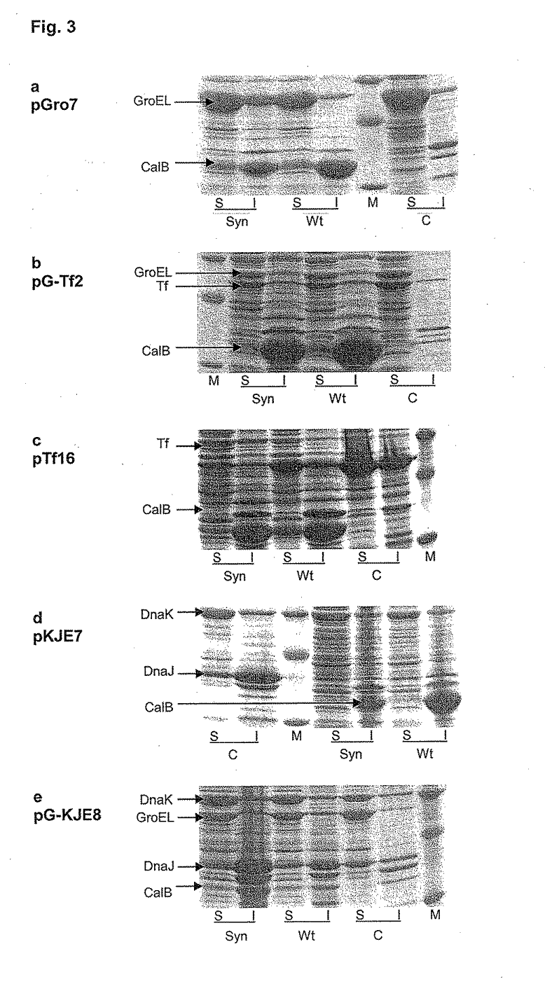

All recombinant Origami™ B cells, bearing both CalB and chaperone-expression plasmids, showed at an incubation temperature of 15° C. clear halo formation on tributyrin-agar plates and expression of CalB at the shaken-flask scale (FIG. 3), whereas the amount of soluble lipase varied markedly (FIG. 4). CalB_wt was expressed the most efficiently on expression together with pGro7 (61 U/mg) (FIG. 4). The expression of the functional enzyme was also increased through expression together with pG-Tf2 (33 U/mg), pTf16 (24 U/mg) and, to a smaller extent, through expression together with pG-KJE8 (18 U/mg). Co-expression together with pKJE7 did not have a significant influence on expression of CalB. The results of the activity assay were confirmed by densitometry, with the largest amount of soluble CalB being found in co-expression with pGro7, followed by co-expression with pG-Tf2 and pTf16 (Table 3).

Similar results were obtained with the synthetic calB gene, thus showing a positive influence of the co-expression of pGro7, pG-Tf2, pTf16 and pG-KJE8. As in pET-32b(+) and pColdIII expression, the values obtained for lipase activities and content of soluble enzyme were lower with the synthetic gene calB_syn than with the wild-type gene calB_wt (FIG. 4, Table 3).

4. Lipase Expression on Tributyrin-Agar Plates

GroES and GroEL (encoded by pGro7) and calB_wt or calB_syn expressing Origami™ B cells showed, on incubation at 15° C., clear halo formation on tributyrin-supplemented agar plates. Halo formation was not observed for DH5α cells that contained the same constructs, nor for Origami™ B control cells that contained pGro7 vector and pColdIII vector without the lipase gene (data not shown).

5. Lipase Expression at the Microtiter Plate Scale

As a model for a high-throughput screening system for enzyme variants derived from CalB, the co-expression of calB_wt or calB_syn with pGro7 was carried out in Origami™ B cells at the scale of a 96-well microtiter plate. The activities of the clarified cell lysates were quantified by calorimetric assay using pNPP as substrate. As shown by the formation of yellow p-nitrophenolate (FIG. 6), both constructs were expressed functionally in comparable amounts. Control cells without the lipase gene showed significantly reduced extinction values at 410 nm.

To compare the activities in expression in microtiter plates with the values that were obtained with co-expression of the constructs pColdIII/calB_wt and pGro7 in shaken flasks, the activities of 5 representative wells with the substrate tributyrin were investigated by means of the pH Stat assay. The growing conditions were identical in the individual wells. The results found were specific activities from 57 to 81 U/mg soluble protein in the clarified cell lysate or total activities from 10 to 15 U/ml cell culture. The expression level of total lipase in the wells was determined by densitometric analysis as 0.04±0.01 μg CalB/ml cell culture.

### REFERENCES

Anderson E. M., Larsson, K. M., Kirk, O., Biocat. Biotransform. 16: 181-204 (1998)

Ausubel et al., Current Protocols in Molecular Biology, John Wiley & Sons, N.Y., 1989

Baneyx F., Mujacic M., Nature Biotechnol. 22 (11): 1399-408 (2004)

Boel E., Huge-Jensen B., Christensen M., Thim L., Fill N. P., Lipids 23:701-706 (1988)

Bradford M. M., Anal. Biochem. 72: 248-254 (1976)

Chodrorge M., Fourage L., Ullmann C., Duvivier V., Masson J. M., Lefèvre F., Adv. Synth. Catal. 347: 1022-1026 (2005)

Cooper T. G., “Biochemische Arbeitsmethoden”, Walter de Gruyter-Verlag, Berlin, 1. Aufl., 1980, Seite 334-379

Giacalone M. J., Gentile A. M., Lovitt B. T., Berkley N. L., Gunderson C. W., Surber M. W, Biotechniques 40 (3): 355-364 (2006)

Giladi H., Goldenberg D., Koby S., Oppenheim A. B., Proc Natl Acad Sci USA 92:184-188 (1995)

Goeddel, Gene Expression Technology: Methods in Enzymology 185, Academic Press, San Diego, Calif. (1990)

Goldstein J., Pollitt N. S., Inouye M., Proc. Natl. Acad. Sci. USA 87 (1): 283-7 (1990)

Hale R. S., Thompson, G., Protein Expr. Purif. 12: 185-188 (1988)

Hoegh I., Patkar, S., Halkier, T., Hansen, M. T., Can. J. Bot. 73: 869-875 (1995)

Ike Y, Ikuta S, Sato M, Huang T, Itakura K., Nucleic Acids Res. 11:477-488 (1983)

Ikura K., Kokubu T., Natsuka S., Ichikawa A., Adachi M., Nishihara K., Yanagi H., Utsumi S., Prep. Biochem. Biotechnol. 32 (2): 189-205 (2002)

Itakura K, Rossi J. J., Wallace R. B., Annu. Rev. Biochem. 53:323-356 (1984)

Itakura K, Hirose T, Crea R, Riggs A D, Heyneker H L, Bolivar F, Boyer H W, Science 198:1056-1063 (1984)

Kirchhoff F., Desrosiers R. C., Methods Mol Biol. 57:323-33 (1996)

Laemmli U. K., Nature 227: 680-685 (1970)

Longhi S., Fusetti F., Grandori R., Lotti M., Vanoni M., Alberghina L., Biochim Biophys Acta 1131:227-232 (1992)

Lutz R., Bujard H., Nucleic Acids Res. 25 (6): 1023-1210 (1997)

Martinelle M., Holmquist M., Hult K., Biochim. Biophys. Acta 1258 (3): 272-6 (1995)

Nakashima K., Kanamaru K., Mizuno T., Horikoshi K., J. Bacteriol. 178:2994-2997 (1996)

Narang, S. A., Tetrahedron 39:3 (1983)

Nishihara K., Kanemori M., Yanagi H., Yura T., Appl. Environ. Microbiol. 66 (3): 884-9 (2000)

Novagen (1999) pET Sytem Manual

Ollis D. L., Cheah, E., Cygler, M., Dijkstra, B., Frolow, F., Franken, S. M., Harel, M., Remingon, S. M., Silman, L., Schrag, J. D., Protein Eng. 5: 197-211 (1992).

Patkar S., Vind J., Kelstrup E., Christensen M. W., Svendsen A., Borch K., Kirk O., Chem. Phys. Lipids 93 (1-2): 95-101 (1988)

Pearson W. R. und Lipman, D. J., Proc. Natl. Acad, Sci. (USA) 85 (8): 2444-2448 (1988)

Prinz W. A., Aslund F., Holmgren A., Beckwith J. J. Biol. Chem. 272 (25): 15661-7 (1997)

Qing G., Ma L.-C., Khorchid A., Swapna G. V. T., Mal T. K., Takayama M. M., Xia B., Phadtare S., Ke H., Acton T., Montelione G., Ikura M. und. Inouye M., Nature Biotechnol. 22 (7):877-882 (2004)

Qoronfleh M. W., Debouck C. Keller J., J. Bacterial. 174:7902-7909 (1992)

Rotticci D. “Understanding and Engineering the Enantioselectivity of Candida antarctica Lipase B towards sec-Alcohols”. Stockholm: Royal institute of Technology 1-61 (2000)

Rotticci-Mulder J. C., “Expression and mutagenesis studies of Candida antarctica lipase B [PhD]”. Stockholm: AlbaNova University Centre 1-74 (2003)

Rotticci-Mulder J. C., Gustavsson M., Holmquist M., Hult K., Martinelle M., Protein Expr. Purif. 21 (3): 386-92 (2001)

Roufa D. J., Methods Mol. Biol. 57: 357-367 (1996)

Rusnak M. “Untersuchungen zur enzymatischen Enantiomerentrennung von Glykolethern und Etablierung neuer Methoden des synthetischen Shufflings”. Universität Stuttgart (2004)

Schmid R. D., Verger, R., Angew. Chem. Int. Ausg. 37: 1608-33 (1998)

Schofield D. A., Westwater C., Dolan J. W., Norris J. S., Schmidt M. G., Curr. Microbiol. 44 (6): 425-430 (2002)

Scopes R. “Protein Purification. Prinicples and Practise”. Springer Verlag, New York, 1. Aufl., 1981

Suen, W. C., Zhang, N., Xiao, L, Madison, V., Zaks, A. Protein Eng. Des. Sel. 17 (2): 133-40 (2004)

TaKaRa (2003) Cold Shock Expression System pCold DNA, Manual 1-9

TaKaRa (2003b) Chaperone Plasmid Set Manual 1-7

Tsuchiya A., Nakazawa H., Toida J, Ohnishi K, Sekiguchi J, FEMS Microbiol Lett. 143:63-7 (1996)

Uppenberg J., Hansen, M. T., Patkar, S., Jones, A., Structure 2: 293-308 (1994)

Yamaguchi S., Mase T., Takeuchi K., Gene 103:61-67 (1991)

Zhang N., Suen W. C., Windsor W., Xiao L., Madison V., Zaks A., Protein Eng. 16 (8): 599-605 (2003)

The following sequences or plasmids, referred to in the above description, are included in the sequence listing under the stated SEQ ID NOs:

calB_syn: SEQ ID NO:1

calB_wt: SEQ ID NO:2

Promoter of T7: SEQ ID NO:3

cspA promoter (E. coli): SEQ ID NO:4

calB_syn in pUC19 (pUC18/calB_syn): SEQ ID NO:5

calB_wt in pUC19 (pUC18/calB_swt): SEQ ID NO:6

calB_syn in pET-32b(+) (pET-32b(+)/calB_syn): SEQ ID NO:7

calB_wt in pET-32b(+) (pET-32b(+)/calB_wt): SEQ ID NO:8

calB_syn in pCOLDIII (pCOLDIII/calB_syn): SEQ ID NO:9

calB_wt in pCOLDIII (pCOLDIII/calB_wt): SEQ ID NO:10

pPCR/CalB: SEQ ID NO:11

CalB (CAA83122.1): SEQ ID NO:12

CalB with exchange of T57A and A89T: SEQ ID NO:13

1. A method of expressing a functional triacylglycerol lipase (E.C. 3.1.1.3) in prokaryotes, comprising expressing a nucleotide sequence encoding triacylglycerol lipase in a prokaryotic host cell under the control of an inducible promoter at a temperature from 1° C. to 25° C.

2. The method of claim 1, wherein the temperature is from 1° C. to 20° C.

3. The method of claim 2, wherein the temperature is from 1° C. to 17° C.

4. The method of claim 1, wherein the expression takes place in a thioredoxin-reductase-deficient and glutathione-reductase-deficient E. coli strain.

5. The method of claim 1, wherein the nucleotide sequence encoding triacylglycerol lipase encodes a lipase B from Candida antarctica (calB).

6. The method of claim 5, wherein the lipase B is encoded by the nucleotide sequence of SEQ ID NO: 1 (calB_syn) or SEQ ID NO: 2 (calB_wt), or a nucleotide sequence homologous thereto.

7. The method of claim 1, wherein the nucleotide sequence encoding triacylglycerol lipase is under the control of the promoter of T7 (SEQ ID NO: 3).

8. The method of claim 1, wherein the nucleotide sequence encoding triacylglycerol lipase is under the control of a cold-shock-inducible promoter.

9. The method of claim 8, wherein the cold-inducible promoter is the promoter of the cspA gene of E. coli (SEQ ID NO: 4).

10. The method of claim 1, further comprising co-expressing one or more chaperones.

11. The method of claim 10, wherein the one or more chaperones are selected from the group consisting of GroES, GroEL, DnaK, DnaJ, GrpE and Trigger Factor (TF) of E. coli.

12. The method of claim 11, wherein GroEL and GroES are co-expressed.

13. The method of claim 11, wherein DnaK, DnaJ and GrpE are co-expressed.

14. The method of claim 11, wherein Trigger Factor, optionally together with GroES and GroEL, is co-expressed.

15. The method of claim 11, wherein DnaK, DnaJ, GrpE, GroES and GroEL are co-expressed.

16. A method for detecting a triacylglycerol lipase, comprising
i) expressing a protein that is presumed to have triacylglycerol lipase activity according to the method of claim 1,
ii) bringing the expression product into contact with a substrate that is hydrolyzable by a triacylglycerol lipase, and
iii) determining the hydrolysis activity.

17. The method of claim 16, wherein the expression of the protein and/or the determination of its hydrolysis activity are carried out on a microtiter plate.

18. The method of claim 16, wherein the expression of the protein according to step i) takes place in the presence of a substrate that is hydrolyzable by the lipase.

19. The method of claim 18, wherein the expression of the protein takes place on a medium that contains a substrate that is hydrolyzable by the lipase.

20. The method of claim 16, further comprising
i) separating the expressed protein from the cell culture,
ii) contacting the expressed protein with a substrate that is hydrolyzable by a triacylglycerol lipase, and
iii) determining the hydrolysis activity.

21. The method of claim 20, wherein the hydrolysis activity is determined photometrically from the decrease in hydrolyzable substrate or the increase in hydrolysis product.

22. A method for screening proteins having triacylglycerol lipase activity, comprising using the method of claim 16 to determine the hydrolysis activity of the protein, wherein the protein is a mutagenized protein, or a protein that is encoded by a mutagenized nucleic acid.

23. The method of claim 22, wherein the protein has at least one mutation in the coding nucleotide sequence of a lipase B from Candida antarctica (calB).

24. The method of claim 16, wherein the method is carried out as a high-throughput screening method.

25. A method of producing a triacylglycerol lipase (E.C. 3.1.1.3), comprising
i) expressing and detecting a protein having activity of a triacylglycerol lipase (E.C. 3.1.1.3) using the method of claim 16,
ii) cultivating the prokaryotic host cell expressing the protein under lipase-expressing conditions, and
iii) optionally isolating the expressed lipase.

26. A nucleic acid encoding a triacylglycerol lipase, comprising the nucleotide sequence of SEQ ID NO: 1, or a nucleotide sequence homologous thereto in which no more than 25% of the nucleotides are different.

27. A recombinant vector comprising the nucleic acid of claim 26 operatively linked to at least one regulating nucleic acid sequence.

28. A recombinant host cell comprising the nucleic acid of claim 26 and/or a recombinant vector comprising said nucleic acid.

29. (canceled)

30. A method of expressing a functional triacylglycerol lipase (E.C. 3.1.1.3) in prokaryotes, comprising expressing a nucleotide sequence encoding triacylglycerol lipase in a prokaryotic host cell under the control of an inducible promoter at a temperature from 1° C. to 25° C., wherein the nucleic acid of claim 26, or a vector comprising said nucleic acid, or a host cell comprising said nucleic acid or said vector is used for expressing the functional triacylglycerol lipase.


SEQUENCE LISTING (24 sequences)

SEQ ID NO: 1 - DNA, 957 bp, Artificial sequence
  modified from Candida antarctica
ctgccgagcggtagcgatccggcgtttagccagccgaaaagcgttctggatgcgggtctgacctgtcagggtgcgagcccgagcagcgttagcaaaccgattctgctggttccgggcaccggcaccaccggtccgcagagctttgatagcaactggattccgctgagcacccagctgggttataccccgtgttggattagcccgccgccgtttatgctgaatgatacccaggtgaacaccgaatatatggtgaacgcgatcaccgcgctgtatgcgggtagcggcaataataaactgccggtgctgacctggagccagggtggtctggttgcgcagtggggtctgaccttttttccgagcattcgcagcaaagtggatcgtctgatggcgtttgcgccggattataaaggcaccgttctggcgggtccgctggatgcgctggcggttagcgcgccgagcgtttggcagcagaccaccggtagcgcgctgaccaccgcgctgcgtaatgcgggtggtctgacccagattgttccgaccaccaatctgtatagcgcgaccgatgaaattgttcagccgcaggttagcaatagcccgctggatagcagctacctgttcaacggcaaaaatgttcaggcgcaggcggtttgtggtccgctgtttgtgattgatcatgcgggtagcctgaccagccagtttagctatgtggttggtcgtagcgcgctgcgtagcaccaccggccaggcgcgtagcgcggattatggtatcaccgattgcaatccgctgccggcgaatgatctgaccccggaacagaaagttgcggcggcagcgctgctggcgccggcagcggcggccattgttgcgggtccgaaacagaattgtgaaccggatctgatgccgtatgcgcgtccgtttgcggttggtaaacgtacctgcagcggtattgtgaccccgtaataa

SEQ ID NO: 2 - DNA, 954 bp, Candida antarctica
  misc_feature (1)..(3): first coding base triplet after start codon
ctaccttccggttcggaccctgccttttcgcagcccaagtcggtgctcgatgcgggtctgacctgccagggtgcttcgccatcctcggtctccaaacccatccttctcgtccccggaaccggcaccacaggtccacagtcgttcgactcgaactggatccccctctctgcgcagctgggttacacaccctgctggatctcacccccgccgttcatgctcaacgacacccaggtcaacacggagtacatggtcaacgccatcaccacgctctacgctggttcgggcaacaacaagcttcccgtgctcacctggtcccagggtggtctggttgcacagtggggtctgaccttcttccccagtatcaggtccaaggtcgatcgacttatggcctttgcgcccgactacaagggcaccgtcctcgccggccctctcgatgcactcgcggttagtgcaccctccgtatggcagcaaaccaccggttcggcactcactaccgcactccgaaacgcaggtggtctgacccagatcgtgcccaccaccaacctctactcggcgaccgacgagatcgttcagcctcaggtgtccaactcgccactcgactcatcctacctcttcaacggaaagaacgtccaggcacaggctgtgtgtgggccgctgttcgtcatcgaccatgcaggctcgctcacctcgcagttctcctacgtcgtcggtcgatccgccctgcgctccaccacgggccaggctcgtagtgcagactatggcattacggactgcaaccctcttcccgccaatgatctgactcccgagcaaaaggtcgccgcggctgcgctcctggcgccggcggctgcagccatcgtggcgggtccaaagcagaactgcgagcccgacctcatgccctacgcccgcccctttgcagtaggcaaaaggacctgctccggcatcgtcaccccctga

SEQ ID NO: 3 - DNA, 17 bp, Artificial sequence
  derived from pET32b+
taatacgactcactata

SEQ ID NO: 4 - DNA, 67 bp, Artificial sequence
  derived from cspA (E.coli)
ccgattaatcataaatatgaaaaataattgttgcatcacccgccaatgcgtggcttaatgcacatca

SEQ ID NO: 5 - DNA, 3383 bp, Artificial sequence
  calB_syn cloned into pUC18
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgctgccgagcggtagcgatccggcgtttagccagccgaaaagcgttctggatgcgggtctgacctgtcagggtgcgagcccgagcagcgttagcaaaccgattctgctggttccgggcaccggcaccaccggtccgcagagctttgatagcaactggattccgctgagcacccagctgggttataccccgtgttggattagcccgccgccgtttatgctgaatgatacccaggtgaacaccgaatatatggtgaacgcgatcaccgcgctgtatgcgggtagcggcaataataaactgccggtgctgacctggagccagggtggtctggttgcgcagtggggtctgaccttttttccgagcattcgcagcaaagtggatcgtctgatggcgtttgcgccggattataaaggcaccgttctggcgggtccgctggatgcgctggcggttagcgcgccgagcgtttggcagcagaccaccggtagcgcgctgaccaccgcgctgcgtaatgcgggtggtctgacccagattgttccgaccaccaatctgtatagcgcgaccgatgaaattgttcagccgcaggttagcaatagcccgctggatagcagctacctgttcaacggcaaaaatgttcaggcgcaggcggtttgtggtccgctgtttgtgattgatcatgcgggtagcctgaccagccagtttagctatgtggttggtcgtagcgcgctgcgtagcaccaccggccaggcgcgtagcgcggattatggtatcaccgattgcaatccgctgccggcgaatgatctgaccccggaacagaaagttgcggcggcagcgctgctggcgccggcagcggcggccattgttgcgggtccgaaacagaattgtgaaccggatctgatgccgtatgcgcgtccgtttgcggttggtaaacgtacctgcagcggtattgtgaccccgtaataacatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 6 - DNA, 3333 bp, Artificial sequence
  calB_wt cloned into pUC18
gcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgctaccttccggttcggaccctgccttttcgcagcccaagtcggtgctcgatgcgggtctgacctgccagggtgcttcgccatcctcggtctccaaacccatccttctcgtccccggaaccggcaccacaggtccacagtcgttcgactcgaactggatccccctctctgcgcagctgggttacacaccctgctggatctcacccccgccgttcatgctcaacgacacccaggtcaacacggagtacatggtcaacgccatcaccacgctctacgctggttcgggcaacaacaagcttcccgtgctcacctggtcccagggtggtctggttgcacagtggggtctgaccttcttccccagtatcaggtccaaggtcgatcgacttatggcctttgcgcccgactacaagggcaccgtcctcgccggccctctcgatgcactcgcggttagtgcaccctccgtatggcagcaaaccaccggttcggcactcactaccgcactccgaaacgcaggtggtctgacccagatcgtgcccaccaccaacctctactcggcgaccgacgagatcgttcagcctcaggtgtccaactcgccactcgactcatcctacctcttcaacggaaagaacgtccaggcacaggctgtgtgtgggccgctgttcgtcatcgaccatgcaggctcgctcacctcgcagttctcctacgtcgtcggtcgatccgccctgcgctccaccacgggccaggctcgtagtgcagactatggcattacggactgcaaccctcttcccgccaatgatctgactcccgagcaaaaggtcgccgcggctgcgctcctggcgccggcggctgcagccatcgtggcgggtccaaagcagaactgcgagcccgacctcatgccctacgcccgcccctttgcagtaggcaaaaggacctgctccggcatcgtcaccccctaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaga

SEQ ID NO: 7 - DNA, 6835 bp, Artificial sequence
  calB_syn cloned into pET32b+
atgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggttctggttctggccatatgcaccatcatcatcatcattcttctggtctggtgccacgcggttctggtatgaaagaaaccgctgctgctaaattcgaacgccagcacatggacagcccagatctgggtaccgacgacgacgacaaggccatggcgatatcggatccgaattcgctgccgagcggtagcgatccggcgtttagccagccgaaaagcgttctggatgcgggtctgacctgtcagggtgcgagcccgagcagcgttagcaaaccgattctgctggttccgggcaccggcaccaccggtccgcagagctttgatagcaactggattccgctgagcacccagctgggttataccccgtgttggattagcccgccgccgtttatgctgaatgatacccaggtgaacaccgaatatatggtgaacgcgatcaccgcgctgtatgcgggtagcggcaataataaactgccggtgctgacctggagccagggtggtctggttgcgcagtggggtctgaccttttttccgagcattcgcagcaaagtggatcgtctgatggcgtttgcgccggattataaaggcaccgttctggcgggtccgctggatgcgctggcggttagcgcgccgagcgtttggcagcagaccaccggtagcgcgctgaccaccgcgctgcgtaatgcgggtggtctgacccagattgttccgaccaccaatctgtatagcgcgaccgatgaaattgttcagccgcaggttagcaatagcccgctggatagcagctacctgttcaacggcaaaaatgttcaggcgcaggcggtttgtggtccgctgtttgtgattgatcatgcgggtagcctgaccagccagtttagctatgtggttggtcgtagcgcgctgcgtagcaccaccggccaggcgcgtagcgcggattatggtatcaccgattgcaatccgctgccggcgaatgatctgaccccggaacagaaagttgcggcggcagcgctgctggcgccggcagcggcggccattgttgcgggtccgaaacagaattgtgaaccggatctgatgccgtatgcgcgtccgtttgcggttggtaaacgtacctgcagcggtattgtgaccccgtaagcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacat

SEQ ID NO: 8 - DNA, 6835 bp, Artificial sequence
  calB_wt cloned into pET32b+
atgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggttctggttctggccatatgcaccatcatcatcatcattcttctggtctggtgccacgcggttctggtatgaaagaaaccgctgctgctaaattcgaacgccagcacatggacagcccagatctgggtaccgacgacgacgacaaggccatggcgatatcggatccgaattcgctaccttccggttcggaccctgccttttcgcagcccaagtcggtgctcgatgcgggtctgacctgccagggtgcttcgccatcctcggtctccaaacccatccttctcgtccccggaaccggcaccacaggtccacagtcgttcgactcgaactggatccccctctctgcgcagctgggttacacaccctgctggatctcacccccgccgttcatgctcaacgacacccaggtcaacacggagtacatggtcaacgccatcaccacgctctacgctggttcgggcaacaacaagcttcccgtgctcacctggtcccagggtggtctggttgcacagtggggtctgaccttcttccccagtatcaggtccaaggtcgatcgacttatggcctttgcgcccgactacaagggcaccgtcctcgccggccctctcgatgcactcgcggttagtgcaccctccgtatggcagcaaaccaccggttcggcactcactaccgcactccgaaacgcaggtggtctgacccagatcgtgcccaccaccaacctctactcggcgaccgacgagatcgttcagcctcaggtgtccaactcgccactcgactcatcctacctcttcaacggaaagaacgtccaggcacaggctgtgtgtgggccgctgttcgtcatcgaccatgcaggctcgctcacctcgcagttctcctacgtcgtcggtcgatccgccctgcgctccaccacgggccaggctcgtagtgcagactatggcattacggactgcaaccctcttcccgccaatgatctgactcccgagcaaaaggtcgccgcggctgcgctcctggcgccggcggctgcagccatcgtggcgggtccaaagcagaactgcgagcccgacctcatgccctacgcccgcccctttgcagtaggcaaaaggacctgctccggcatcgtcaccccctgagcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacat

SEQ ID NO: 9 - DNA, 5307 bp, Artificial sequence
  calB_syn cloned into pCOLDIII
atgctgccgagcggtagcgatccggcgtttagccagccgaaaagcgttctggatgcgggtctgacctgtcagggtgcgagcccgagcagcgttagcaaaccgattctgctggttccgggcaccggcaccaccggtccgcagagctttgatagcaactggattccgctgagcacccagctgggttataccccgtgttggattagcccgccgccgtttatgctgaatgatacccaggtgaacaccgaatatatggtgaacgcgatcaccgcgctgtatgcgggtagcggcaataataaactgccggtgctgacctggagccagggtggtctggttgcgcagtggggtctgaccttttttccgagcattcgcagcaaagtggatcgtctgatggcgtttgcgccggattataaaggcaccgttctggcgggtccgctggatgcgctggcggttagcgcgccgagcgtttggcagcagaccaccggtagcgcgctgaccaccgcgctgcgtaatgcgggtggtctgacccagattgttccgaccaccaatctgtatagcgcgaccgatgaaattgttcagccgcaggttagcaatagcccgctggatagcagctacctgttcaacggcaaaaatgttcaggcgcaggcggtttgtggtccgctgtttgtgattgatcatgcgggtagcctgaccagccagtttagctatgtggttggtcgtagcgcgctgcgtagcaccaccggccaggcgcgtagcgcggattatggtatcaccgattgcaatccgctgccggcgaatgatctgaccccggaacagaaagttgcggcggcagcgctgctggcgccggcagcggcggccattgttgcgggtccgaaacagaattgtgaaccggatctgatgccgtatgcgcgtccgtttgcggttggtaaacgtacctgcagcggtattgtgaccccgtaagaattcaagcttgtcgacctgcagtctagataggtaatctctgcttaaaagcacagaatctaagatccctgccatttggcggggatttttttatttgttttcaggaaataaataatcgatcgcgtaataaaatctattattatttttgtgaagaataaatttgggtgcaatgagaatgcgcaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgtggccgattaatcataaatatgaaaaataattgttgcatcacccgccaatgcgtggcttaatgcacatcaaattgtgagcggataacaatttgatgtgctagcgcatatccagtgtagtaaggcaagtcccttcaagagttatcgttgatacccctcgtagtgcacattcctttaacgcttcaaaatctgtaaagcacgccatatcgccgaaaggcacacttaattattaagaggtaatacaccatgaatcacaaagtgcat

SEQ ID NO: 10 - DNA, 5307 bp, Artificial sequence
  calB_wt cloned into pCOLDIII
atgctaccttccggttcggaccctgccttttcgcagcccaagtcggtgctcgatgcgggtctgacctgccagggtgcttcgccatcctcggtctccaaacccatccttctcgtccccggaaccggcaccacaggtccacagtcgttcgactcgaactggatccccctctctgcgcagctgggttacacaccctgctggatctcacccccgccgttcatgctcaacgacacccaggtcaacacggagtacatggtcaacgccatcaccacgctctacgctggttcgggcaacaacaagcttcccgtgctcacctggtcccagggtggtctggttgcacagtggggtctgaccttcttccccagtatcaggtccaaggtcgatcgacttatggcctttgcgcccgactacaagggcaccgtcctcgccggccctctcgatgcactcgcggttagtgcaccctccgtatggcagcaaaccaccggttcggcactcactaccgcactccgaaacgcaggtggtctgacccagatcgtgcccaccaccaacctctactcggcgaccgacgagatcgttcagcctcaggtgtccaactcgccactcgactcatcctacctcttcaacggaaagaacgtccaggcacaggctgtgtgtgggccgctgttcgtcatcgaccatgcaggctcgctcacctcgcagttctcctacgtcgtcggtcgatccgccctgcgctccaccacgggccaggctcgtagtgcagactatggcattacggactgcaaccctcttcccgccaatgatctgactcccgagcaaaaggtcgccgcggctgcgctcctggcgccggcggctgcagccatcgtggcgggtccaaagcagaactgcgagcccgacctcatgccctacgcccgcccctttgcagtaggcaaaaggacctgctccggcatcgtcaccccctgagaattcaagcttgtcgacctgcagtctagataggtaatctctgcttaaaagcacagaatctaagatccctgccatttggcggggatttttttatttgttttcaggaaataaataatcgatcgcgtaataaaatctattattatttttgtgaagaataaatttgggtgcaatgagaatgcgcaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgtggccgattaatcataaatatgaaaaataattgttgcatcacccgccaatgcgtggcttaatgcacatcaaattgtgagcggataacaatttgatgtgctagcgcatatccagtgtagtaaggcaagtcccttcaagagttatcgttgatacccctcgtagtgcacattcctttaacgcttcaaaatctgtaaagcacgccatatcgccgaaaggcacacttaattattaagaggtaatacaccatgaatcacaaagtgcat

SEQ ID NO: 11 - DNA, 3836 bp, Artificial sequence
  pPCR/calB
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgaattcgctgccgagcggtagcgatccggcgtttagccagccgaaaagcgttctggatgcgggtctgacctgtcagggtgcgagcccgagcagcgttagcaaaccgattctgctggttccgggcaccggcaccaccggtccgcagagctttgatagcaactggattccgctgagcacccagctgggttataccccgtgttggattagcccgccgccgtttatgctgaatgatacccaggtgaacaccgaatatatggtgaacgcgatcaccgcgctgtatgcgggtagcggcaataataaactgccggtgctgacctggagccagggtggtctggttgcgcagtggggtctgaccttttttccgagcattcgcagcaaagtggatcgtctgatggcgtttgcgccggattataaaggcaccgttctggcgggtccgctggatgcgctggcggttagcgcgccgagcgtttggcagcagaccaccggtagcgcgctgaccaccgcgctgcgtaatgcgggtggtctgacccagattgttccgaccaccaatctgtatagcgcgaccgatgaaattgttcagccgcaggttagcaatagcccgctggatagcagctacctgttcaacggcaaaaatgttcaggcgcaggcggtttgtggtccgctgtttgtgattgatcatgcgggtagcctgaccagccagtttagctatgtggttggtcgtagcgcgctgcgtagcaccaccggccaggcgcgtagcgcggattatggtatcaccgattgcaatccgctgccggcgaatgatctgaccccggaacagaaagttgcggcggcagcgctgctggcgccggcagcggcggccattgttgcgggtccgaaacagaattgtgaaccggatctgatgccgtatgcgcgtccgtttgcggttggtaaacgtacctgcagcggtattgtgaccccgtaataacatatgcgagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 12 - protein, 317 aa, Candida antarctica [sequence omitted]

SEQ ID NO: 13 - protein, 317 aa, Candida antarctica
  MISC_FEATURE: mature protein CAA83122 (without signal sequence) with exchange of T57A and A89T
LPSGSDPAFSQPKSVLDAGLTCQGASPSSVSKPILLVPGTGTTGPQSFDSNWIPLSAQLGYTPCWISPPPFMLNDTQVNTEYMVNAITTLYAGSGNNKLPVLTWSQGGLVAQWGLTFFPSIRSKVDRLMAFAPDYKGTVLAGPLDALAVSAPSVWQQTTGSALTTALRNAGGLTQIVPTTNLYSATDEIVQPQVSNSPLDSSYLFNGKNVQAQAVCGPLFVIDHAGSLTSQFSYVVGRSALRSTTGQARSADYGITDCNPLPANDLTPEQKVAAAALLAPAAAAIVAGPKQNCEPDLMPYARPFAVGKRTCSGIVTP

SEQ ID NO: 14 - DNA, 29 bp, Artificial sequence
  Primer
gatgaattcgctaccttccggttcggacc

SEQ ID NO: 15 - DNA, 27 bp, Artificial sequence
  Primer
ccacatatgtcagggggtgacgatgcc

SEQ ID NO: 16 - DNA, 24 bp, Artificial sequence
  Primer
ccggaattcgctaccttccggttc

SEQ ID NO: 17 - DNA, 27 bp, Artificial sequence
  Primer
cggcatcgtcaccccctaagcggccgc

SEQ ID NO: 18 - DNA, 30 bp, Artificial sequence
  Primer
cgattcatatgctaccttccggttcggacc

SEQ ID NO: 19 - DNA, 30 bp, Artificial sequence
  Primer
ccttaagaattctcagggggtgacgatgcc

SEQ ID NO: 20 - DNA, 21 bp, Artificial sequence
  Primer
ccggaattcgctgccgagcgg

SEQ ID NO: 21 - DNA, 33 bp, Artificial sequence
  Primer
gtattgtgaccccgtaataacatatggaattcc

SEQ ID NO: 22 - DNA, 27 bp, Artificial sequence
  Primer
gcggtattgtgaccccgtaagcttggg

SEQ ID NO: 23 - DNA, 29 bp, Artificial sequence
  Primer
cagttcatatgctgccgagcggtagcgat

SEQ ID NO: 24 - DNA, 33 bp, Artificial sequence
  Primer
ccttaagaattcttacggggtcacaataccgct